(19) United States
(12) Patent Application Publication
(10) Pub. No.: US 2025/0215423 A1
(43) Pub. Date: Jul. 3, 2025

(54) ONE VECTOR SYSTEM FOR IDENTIFICATION OF GENOME MODIFYING ENZYMES

(71) Applicant: Beam Therapeutics Inc., Cambridge, MA (US)

(72) Inventor: Zharko DANILOSKI, Cambridge, MA (US)

(21) Appl. No.: 19/079,571

(22) Filed: Mar. 14, 2025

Related U.S. Application Data
(63) Continuation of application No. PCT/US2023/074297, filed on Sep. 15, 2023.
(60) Provisional application No. 63/407,494, filed on Sep. 16, 2022.

(51) Int. Cl. C12N 15/10; C12N 9/22; C12N 15/86; C12Q 1/6869

(52) U.S. Cl. CPC C12N 15/1082; C12N 9/22; C12N 15/1086; C12N 15/86; C12Q 1/6869; C12Y 301/22

(57) ABSTRACT

The present application relates to an integrated one vector system for identifying a genome modifying enzyme, e.g., from a large library. The one-vector system integrates the components for enzyme identification into a single vector to facilitate a high throughput screening process.

### CROSS REFERENCE TO RELATED APPLICATIONS

This application is a Continuation Application of International Application No. PCT/US2023/074297, filed on Sep. 15, 2023, which claims priority to U.S. Provisional Patent Application No. 63/407,494, filed on Sep. 16, 2022, the contents of each of which are incorporated by reference herein in entirety for all purposes.

### BACKGROUND

Genome modifying enzymes such as recombinases, CRISPR/Cas nucleases and other enzymes are very useful tools for gene function analysis, gene transformation and gene therapy.

Many systems and assays have been used to identify an active genome modifying enzyme. They rely on commonly used two-vectors system in which, candidate genome modifying enzymes are expressed in one vector (e.g., enzyme expressing vector), while the enzyme specific recognition site sequence and other required elements are included in another vector (e.g., donor vector). The two vectors system is not compatible when screening a large library of enzymes (100s to 1000s) in a pooled screen format. The reason is that both the candidate genome modifying enzyme vector and the cognate enzyme specific recognition site sequence vector must be present in the same cell to ascertain whether the candidate enzyme has function.

A simpler and suitable for high-throughput screening system and assay is of need. The present invention provides an integrated one-vector system that is suitable for screening genome modifying enzyme.

### SUMMARY OF THE INVENTION

The present application provides, among other things, an integrated one vector system for identifying a genome modifying enzyme. The present one vector system is simple and suitable for high-throughput screens to identify an active enzyme from numerous enzyme polypeptides predicted through genome sequences and computational sequence analysis and characterization from extended sequence datasets. The one-vector system, in combination with the current sequencing technologies (e.g., NGS) enables screening hundreds to thousands of enzymes in one large experiment.

The one-vector system integrates the components for enzyme identification in a single vector, including the enzyme polypeptide and its specific target site sequence, to facilitate a simple process and high-throughput screening. In particular, the integrated vector can minimize the transfection variations, as compared to the commonly used two-vectors based systems which separate the enzyme polypeptide in an expression vector and the target site sequence specific to each enzyme in a second donor vector, and require co-transfection of the two vectors.

In one aspect of the present invention, a one vector system for identification of a genome modifying enzyme is provided. The one-vector system comprises at least one integrated vector that comprises a polynucleotide encoding a genome modifying enzyme or an enzyme polypeptide, a target site sequence that is recognizable by the genome modifying enzyme, and a unique identifier that correlates to the genome modifying enzyme in the vector.

The genome modifying enzymes include serine recombinases (e.g., large and small serine recombinases), tyrosine recombinases including small and large tyrosine recombinases (e.g., Flp, λ-integrase, and Dre), retrotransposons (e.g., LTRs, LINEs and SINEs), DNA transposases, nucleases, engineered genome modifying enzymes, chimeric genome modifying enzymes, enzyme polypeptides, antibodies, and the like.

The genome modifying enzyme is from any biological system, for example, from bacteriophages, cyanophages, mycoviruses, archaeal viruses, fungi (e.g., yeast), bacteria, and animal microbiome,

In some embodiments, the genome modifying enzyme is a recombinase. The recombinases can be isolated from any bacteriophages, cyanophages, mycoviruses, archaeal viruses, fungi, bacteria, animal microbiome such as human gut microbiome.

In some embodiments, the genome modifying enzyme is a serine recombinase, such as large serine recombinase (LSR) and small serine recombinase. In other embodiments, the genome modifying enzyme is a tyrosine recombinase (TR), such as large tyrosine recombinase and small tyrosine recombinase.

In some embodiments, the genome modifying enzyme is a DNA transposase.

In some embodiments, the genome modifying enzyme or the genome modifying element is a transposable element such as a retrotransposon. As non-limiting examples, the retrotransposons include long terminal repeat (LTR) retrotransposons, long interspersed nuclear element (LINE) retrotransposons and long interspersed nuclear element (SINE) retrotransposons.

In accordance, the integrated vector of a one vector system includes at least one target site that is recognizable by the genome modifying enzyme in the same vector. The target site sequence is a native target site sequence, or an engineered (e.g., mutated) target site sequence that is recognizable by the genome modifying enzyme.

In some embodiments, the target site sequence may include a predicted target site sequence for the genome modifying enzyme in the same vector; the sequence is predicted based on, e.g., characteristics and genome modification activities of the enzyme.

In accordance, the integrated vector comprises a unique based identifier. In some embodiments, the unique identifier is a nucleic acid barcode comprising a nucleotide sequence of about 5-30, 5-20, 8-30, 8-20, 10-30, or 10-20 nucleotides in length. In some embodiments, the unique identifier is a DNA barcode comprising about 20, 18, 16, 12 or 10 nucleotides. The barcode sequence does not affect expression of the encoded enzyme nor its genomic modification activity. In some embodiments, the barcode sequence is randomly generated. In some embodiments, the randomly generated barcode sequences each have at least two, at least three, at least four, at least five, at least six, at least seven, at least eight, or more nucleotide sequence differences from all the other randomly generated barcode sequences.

In one embodiment, the unique identifier comprises a nucleotide sequence of about 20 nucleotides in length.

The vector further comprises a reporter. In some embodiments, the reporter is a fluorescent protein (e.g., GFP or variant thereof, YFP or variant thereof, BFP or variant thereof, CFP or variant thereof and RFP or variant thereof, etc.).

The vector may further comprise a selection marker, such as an antibiotic resistance marker, e.g., to resistant to antibiotics Puromycin, Hygromycin, Blasticidin, Neomycin, Kanamycin, etc.

In some embodiments, the vector comprises a promoter sequence. The promoter is constitutive or inducible. In one exemplary embodiment, the promoter is a CMV promoter.

The vector can be a non-viral vector (e.g., plasmid, cosmid, artificial chromosome vector, etc.) or a viral based vector.

In some embodiments, the viral vector is an adeno-associated viral (AAV) vector such as a recombinant AAV vector, an adenoviral vector, a retroviral vector, a lentiviral vector, a herpesviral vector, a rabies viral vector, or the like.

In some embodiments, the one vector system comprises at least one messenger mRNA comprising a polynucleotide encoding a genome modifying enzyme or an enzyme polypeptide, a target site sequence that is recognizable by the genome modifying enzyme, and a unique identifier that correlates to the genome modifying enzyme. The fused mRNA molecule further comprises other regulatory sequences that facilitate its expression in a host cell and/or organism, for example a 3′ end poly (A) sequence. The fused mRNA molecule can be formulated with a lipid based vehicle for transducing into a host cell and/or organism.

As a non-limiting example, the present invention provides a one vector system for identifying a large serine recombinase; the one vector system comprises at least one integrated vector comprising a polynucleotide encoding a large serine recombinase, a target site sequence that is recognizable by the serine recombinase, and a unique identifier that correlates to the genome modifying enzyme in the vector.

In some embodiments, the target site sequence specific to a large serine recombinase comprises an attP recognition site sequence comprising about 300 base pairs upstream of the LSR coding sequence in the phage genome and about 300 base pairs downstream of the LSR coding sequence in the phage genome. The target site sequence comprises a pseudo attP recognition site sequence that is recognizable by the LSR.

Alternatively, the target site sequence comprises a corresponding attB recognition site sequence or a pseudo attB recognition site sequence that is recognizable by the LSR in the vector.

In a further aspect of the present invention, a method of identifying a genome modifying enzyme using the one-vector system contemplated herein is provided.

The method can be performed in a high-throughput manner where a plurality of integrated vectors, each of which comprises a nucleotide sequence encoding a unique genome modifying enzyme, a target site sequence that is recognizable by the genome modifying enzyme, and a unique identifier that correlates to the genome modifying enzyme, are used. Thereafter, the plurality of integrated vectors can be introduced into a population of hosts for further characterization. Such high-throughput screening can be used to effectively identify an active genome modifying enzyme.

In some embodiments, the method of identifying a genome modifying enzyme comprises: i) transfecting into cells a plurality of vectors, each of which comprises a nucleotide sequence encoding a unique genome modifying enzyme, a target site sequence that is recognizable by the unique genome modifying enzyme in the vector and a unique identifier that correlates to the genome modifying enzyme in the vector; ii) detecting genome modification activities in the transfected cells; iii) identifying the genome modifying enzyme by identifying the unique identifier in the transfected cells in which the genome modification activities are detected.

The cells can be any cells. In some embodiments, mammalian cells are used. In some examples, the cells are human cells. In some embodiments, the cells are cell line cells (e.g., HEK293T cells).

Any known methods for transducing vectors and exogenous nucleic acid molecules into cells can be used (e.g., lipofection, electroporation, etc.).

In some embodiments, the genome modification activities are detected using sequencing technologies, such as next-generation sequencing (NGS).

In yet another aspect of the present invention, a method for identifying a recognition site sequence for a genome modifying enzyme using a one vector system described herein is provided; the method comprises: i) transfecting into cells a plurality of vectors, each of which comprises a polynucleotide encoding the genome modifying enzyme and a unique target site sequence and a unique identifier that correlates to the unique target site sequence; ii) detecting recombination activity in the transfected cells; iii) identifying the recognition site sequence by identifying the unique identifier in the transfected cells in which the genome modification activity is detected.

As a representative example, the method is used to identify the recognition sequence of a large serine recombinase. In this context, each one of the vectors in the system comprises a polynucleotide encoding the same LSR, a unique recognition sequence and a unique identifier that correlates to the unique recognition sequence. The recognition sequence is an attP sequence, a pseudo attP sequence, a mutated attP sequence, an attB sequence, a pseudo attB sequence, or a mutated attB sequence. In some embodiments, the genomic recombination is detected using NGS.

Also provided in the present invention are libraries and kits constructed using the vectors described herein and for carrying out the methods described herein. Further provided are sub-libraries generated during the screening process, and genome modifying enzymes and specific target sequences for an individual enzyme obtained from methods described herein.

### BRIEF DESCRIPTON OF THE DRAWINGS

FIG. 1 is a representative figure for one-vector system. The diagram demonstrates an integrated vector comprising a nucleotide sequence encoding a large serine recombinase, a target site sequence, i.e., the attP sequence, that is recognizable by the LSR, and a unique identifier (i.e., barcode) that correlates to the LSR in the vector.

FIG. 2 is a diagram that demonstrates the screening process to identify a recombinase from an enzyme library.

### DETAILED DESCRIPTION

### Definitions

In order for the present invention to be more readily understood, certain terms are first defined below. Additional definitions for the following terms and other terms are set forth throughout the specification.

The articles “a” and “an” are used herein to refer to one or to more than one (i.e., to at least one) of the grammatical object of the article. By way of example, “an element” means one element or more than one element.

The term “approximately” or “about,” as used herein, as applied to one or more values of interest, refers to a value that is similar to a stated reference value. In certain embodiments, the term “approximately” or “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, when used to define products, compositions and methods, the term “comprising” is intended to mean that the products, compositions and methods include the referenced components or steps, but not excluding others. “Consisting essentially of” shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps. For example, a polypeptide “consists of”' an amino acid sequence when the polypeptide does not contain any amino acids but the recited amino acid sequence. A polypeptide “consists essentially of” an amino acid sequence when such an amino acid sequence is present together with only a few additional amino acid residues, typically from about 1 to about 50 or so additional residues. A polypeptide “comprises” an amino acid sequence when the amino acid sequence is at least part of the final amino acid sequence of the polypeptide. Such a polypeptide can have a few up to several hundred additional amino acids residues. Such additional amino acid residues may play a role in polypeptide trafficking, facilitate polypeptide production or purification; prolong half-life, among other things. The same can be applied for nucleotide sequences.

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progenies may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Enzyme: The term “enzyme” as defined herein encompasses native as well as modified enzymes. The term “native” as used herein refers to a material recovered from a source in nature as distinct from material artificially modified or altered by man in the laboratory. For example, a native enzyme is encoded by a gene that is present in the genome of a wild-type organism or cell. By contrast, a modified or engineered enzyme is encoded by a nucleic acid molecule that has been modified in the laboratory so as to differ from the native polypeptide, e.g., by insertion, deletion or substitution of one or more amino acid(s) or any combination of these possibilities. A genome modifying enzyme refers to any enzyme that can modify a genome in a host organism and/or a host cell.

Genome modification: As used herein, the term “modification” or “modifying” or “modified” when applied to nucleic acid sequences, refers to any change to the sequences within the genome, such as single nucleotide variant (SNV), insertion, deletion, site specific recombination, substitution, chromosomal translocation and structural variation (SV), etc. For example, in terms of insertion, the sequence modification may be the integration of a transgene into a target genomic site. For example, for a target genomic sequence, the donor DNA comprises a sequence complementary, identical, or homologous to the target genomic sequence and a sequence modification region.

Genome modifying enzyme: As used herein, the term “genome modifying enzyme” refers to any protein with catalytic function to modify nucleic acid sequence in a genome, such as any enzyme that binds to a nucleic acid sequence and modifies the sequence. Exemplary genome modifying enzymes include recombinases including serine recombinases (e.g., large and small serine recombinases) and tyrosine recombinases (e.g., small and large tyrosine recombinases), DNA transposases, retrotransposons (e.g., LTRs, LINEs and SINEs), integrases, nucleases, base editors and prime editors, etc. The genome modifying enzymes may also include engineered genome modifying enzymes, chimeric genome modifying enzymes, enzyme polypeptides, and the like.

Genome modifying element: As used herein, the term “genome modifying element” refers to a sequence that can be used to modify nucleic acid sequence in a genome. For example, the genome modifying element can be a transposable element such as a transposon and a retrotransposon. A transposon is transposed by its corresponding transposase to a genome (e.g., a host genome). A transposon is inserted into a target nucleic acid site in the host genome. Retrotransposons (also called Class I transposable elements or transposons via RNA intermediates) are a type of genetic component that copy and paste themselves into different genomic locations (transposon) by converting RNA back into DNA through the reverse transcription process using an RNA transposition intermediate.

Heterologous: As used herein, the term “heterologous”, when used to describe a first element in reference to a second element means that the first element and second element do not exist in nature disposed as described. For example, a heterologous nucleic acid molecule refers to nucleic acid molecule or portion of a nucleic acid molecule sequence that is not native to a cell, or a genome in which it is expressed.

As used herein, the term “homology” or “identity” or “similarity” refers to sequence similarity between two peptides or between two nucleic acid molecules. A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” or “homology” to another sequence means that, when aligned, that percentage of bases (or amino acids) are the same in comparing the two sequences. This alignment and the percent homology or sequence identity can be determined using software programs known in the art.

Mutation: The term “mutation” or “mutated” when applied to nucleic acid sequences means that nucleotides in a nucleic acid sequence may be inserted, deleted or changed compared to a reference (e.g., native) nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art.

mRNA: As used herein, the term “mRNA” refers to messenger RNA which is a polynucleotide that encodes at least one polypeptide.

Nucleoside: As used herein, the term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide,” “oligonucleotide” and “nucleic acid molecule” are used interchangeably herein and refer to a polymer of nucleotides, either deoxyribonucleotides or ribonucleotides, of any length joined together by a phosphodiester linkage between 5′ and 3′ carbon atoms. Oligonucleotide generally is between about 5 and about 100 nucleotides of single-or double-stranded DNA. The terms also refer to both double-and single-stranded molecules. Unless otherwise specified or required, any embodiment of this invention that comprises a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. The terms “RNA,” “RNA molecule” and “ribonucleic acid molecule” refer to a polymer of ribonucleotides. The terms “DNA,” “DNA molecule” and “deoxyribonucleic acid molecule” refer to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. As used herein, the term “isolated RNA” (e.g., “isolated mRNA”) refers to RNA molecules which are substantially free of other cellular material, or culture medium when produced by recombinant techniques, or substantially free of chemical precursors or other chemicals when chemically synthesized.

Within the context of the present invention, the terms “nucleic acid”, “nucleic acid molecule”, “polynucleotide” and “nucleotide sequence” are used interchangeably and define a polymer of any length of either polydeoxyribonucleotide (DNA) or polyribonucleotide (RNA) molecules or any combination thereof. The definition encompasses single or double-stranded, linear or circular, naturally-occurring or synthetic polynucleotides. Moreover, such polynucleotides may comprise non-naturally occurring nucleotides, e.g., methylated nucleotides and nucleotide analogs as well as chemical modifications in order to increase the in vivo stability of the nucleic acid, enhance the delivery thereof, or reduce the clearance rate from the host subject. If present, modifications may be imparted before or after polymerization.

The terms “polypeptide”, “peptide” and “protein” are used herein interchangeably to refer to polymers of amino acid residues which comprise 9 or more amino acids bonded via peptide bonds. The polymer can be linear, branched or cyclic. In the context of this invention, a “polypeptide” may include amino acids that are L stereoisomers (the naturally occurring form) or D stereoisomers and may include amino acids other than the common naturally occurring amino acids, such as [beta]-alanine, ornithine, or methionine sulfoxide, or amino acids modified on one or more alpha-amino, alpha-carboxyl, or side-chain, e.g., by appendage of a methyl, formyl, acetyl, glycosyl, phosphoryl, and the like. As a general indication, if the amino acid polymer is long (e.g., more than 50 amino acid residues), it is preferably referred to as a polypeptide or a protein. By way of consequence, a “peptide” refers to a fragment of about 9 to about 50 amino acids in length. In the context of the invention, a peptide preferably comprises a selected region of a naturally-occurring (or native) protein, e.g. an immunogenic fragment thereof containing an epitope.

Recombination: As used herein the term “recombination” or “recombination reaction” refers to a change of a nucleic acid molecule including, for example, one or more nucleic acid strand breaks (e.g., a double-strand break), followed by joining of two nucleic acid strand ends (e.g., sticky ends). In some instances, the recombination reaction comprises insertion of an insert nucleic acid, e.g., into a target site, e.g., in a genome or a construct. In some instances, the recombination reaction comprises flipping or reversing of a nucleic acid, e.g., in a genome or a construct. In some instances, the recombination reaction comprises removing a nucleic acid, e.g., from a genome or a construct.

Recognition site sequence: A recognition sequence (e.g., DNA recognition sequence) generally refers to a nucleic acid (e.g., DNA) sequence that is recognized (e.g., capable of being bound by) a genome modifying enzyme, e.g., a recombinase. In some embodiments, the recognition sequence is further modified by the bound modifying enzyme. For example, the recognition sequence is cleaved by the bound modifying enzyme. In another example, the recognition sequence is further modified by the bound modifying enzyme by insertion a heterogeneous nucleic acid sequence, or substituting one or more nucleotides.

Safe harbor site (SHS): As used herein, the term “safe harbor site(s) (SHS)” are genomic locations where new genes or genetic elements can be introduced without disrupting the expression or regulation of adjacent genes.

Screening: As used herein, the term “screen” or “screening” refers to the method in which a pool comprising the desired species is subject to an assay in which the desired species can be detected, and subsequently an aliquot of the pool in which the desired species is detected and optionally enriched is recovered or obtained.

The terms “specific” or “specificity” as used herein refers to the property of having a degree of preference for recognizing, binding, hybridizing, recombining, or reacting with a desired target or substrate versus one or more non-desired targets or substrates under the conditions tested or specified. In general, the terms “specific for” or having “specificity for” is used to refer to a preference of at least 50% for the desired target or substrate versus two or more non-desired targets or substrates collectively.

The term “transfected” or “transformed” or “transduced” as used herein refers to a process by which exogenous nucleic acid is transferred or introduced into the host cell. A transformed cell includes the primary subject cell and its progeny. The host cell can be bacteria, yeasts, mammalian cells, and plant cells.

Unique identifier: As used herein, the term “unique identifier” generally refers to any label or identifier such as barcode that can be used to convey information about an agent (e.g., a nucleic acid). An identifier (e.g., barcode) can be a tag or a combination of tags attached to the agent. An identifier can be part of the agent, for example, an internal change to the agent or insertion to the agent. An identifier may be unique. In some embodiments, the unique identifiers are barcodes such as DNA based barcodes or RNA based barcodes. Barcodes can have a variety of different formats, for example, barcodes can include: nucleic acid barcodes; random nucleic acid and/or amino acid sequences; modified nucleic acid barcodes, and synthetic nucleic acid and/or amino acid sequences. The phrases “barcode sequence” and “barcode”, as well as variations thereof, refer to an identifiable nucleotide sequence, such as an oligonucleotide or polynucleotide sequence. In the context of the present disclosure, a barcode is a short nucleic acid, ranging from 4-100, 4-80, 4-60, 4-50, 4-40, 4-30, 6-80, 6-60, 6-40, 8-30, or 8-20 nucleotides in length. A barcode can be a deoxyribonucleic acid (DNA) or ribonucleic acid (RNA) molecule. A barcode is 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, or 30 nucleotides in length. The barcodes can allow for identification and/or quantification of individual sequencing-reads in real time. In some instances, barcodes employed are specially designed with specific unique (i.e., distinct) sequences that are significantly different from each other, even in the case of at least 1 variation. In some embodiments, the barcode sequences in the one-vector system comprise 2, 3, 4, 5, 6, 7, 8 or more different nucleotides from each other. As a non-limiting example, the barcode sequences in the one-vector system comprise 8 different nucleotides from each other.

Vector: As used herein, the term “vector” as used herein refers to a recombinant nucleotide sequence that is capable of effecting expression of a nucleic acid sequence (e.g., transgene) in host cells or host organisms compatible with such sequences. Together with the transgene, expression vectors typically include at least suitable transcript ion regulatory sequences and optionally, 3′ transcript ion termination signals. Additional factors necessary or helpful in effecting expression may also be present, such as expression enhancer elements able to respond to a precise inductive signal (endogenous or chimeric transcription factors) or specific for certain cells, organs or tissues.

Viral vector: A “viral vector” in the present invention refers to a virus particle which lacks self-replication ability and has a capability of introducing a nucleic acid molecule into a host cell.

Various aspects of the invention are described in detail in the following sections. The use of sections is not meant to limit the invention. Each section can apply to any aspect of the invention. In this application, the use of “or” means “and/or” unless stated otherwise

### One-Vector Systems

The present invention relates to one vector systems, among other things, compositions and methods for identifying a genome modifying enzyme. The one-vector system, different from the two-vectors system commonly used in the art, provides a simple system for identification of an active enzyme. In some instances, the present one vector system can allow high throughput screening of large libraries of enzymes isolated or derived from various sources with simplicity and sensitivity. The one-vector screening system can accelerate the identification of a genome modifying enzyme for genome modification such as site specific modification of genome for gene therapy. In other instances, the genome modification activity of a genome modifying enzyme can be characterized using the present one-vector systems, compositions and methods.

In one aspect of the present invention, provided includes a one vector system. The one vector system comprises at least one integrated vector as a key component. In some embodiments, the integrated vector comprises a polynucleotide encoding a genome modifying enzyme, a target site sequence that is recognizable by the genome modifying enzyme, and a unique identifier that correlates to the genome modifying enzyme. In some embodiments, the present one-vector system includes a plurality of such integrated vectors, each of the integrated vectors corresponds to an individual genome modifying enzyme.

### Single Integrated Vector

In accordance, the integrated vector comprises at least one polynucleotide encoding a genome modifying enzyme. The polynucleotide may encode a native enzyme, or an engineered enzyme such as a chimeric enzyme, and a genetically modified or mutated enzyme. As discussed in the following sections, the genome modifying enzyme can be any type of enzyme that has a genome modifying activity such as insertion, deletion, site-specific recombination and translocations.

In some embodiments, the genome modifying enzymes include serine recombinases (e.g., large and small serine recombinases), tyrosine recombinases including small and large tyrosine recombinases (e.g., Flp, λ-integrase, and Dre), retrotransposons (e.g., LTRs, LINEs and SINEs), DNA transposases, nucleases, engineered genome modifying enzymes, chimeric genome modifying enzymes, enzyme polypeptides, and the like.

The genome modifying enzyme is from any biological system, for example, from bacteriophages, cyanophages, mycoviruses, archaeal viruses, and animal microbiome such as human gut microbiome.

The vector, in some instances, comprises one or more enzyme polypeptides. The enzyme polypeptide refers to a polypeptide having the functional capacity to catalyze a genomic modification event (e.g., a recombination reaction) of a nucleic acid molecule (e.g., a DNA molecule). In some embodiments, an enzyme polypeptide comprises one or more structural elements of a naturally occurring enzymes such as the catalytic domains from a naturally occurring enzyme (e.g., nucleases and recombinases). In some embodiments, the enzyme polypeptide comprises an amino acid sequence having at least 70%, at least 75%, at least 80%, at least 85%, at least 90%, at least 95%, at least 96%, at least 97%, at least 98%, at least 99%, or 100% sequence identity to the sequence of a naturally occurring enzyme.

As an illustrative example, the vector comprises a polynucleotide sequence encoding a recombinase or a recombinase polypeptide; the recombinase polypeptide comprises one or more structural elements of a naturally occurring recombinase (e.g., a serine recombinase, such as PhiC31 recombinase and Gin recombinase; or a tyrosine recombination). In certain instances, a recombinase polypeptide comprises an amino acid sequence having at least 70%, at least 75%, at least 80%, at least 85%, at least 90%, at least 95%, at least 96%, at least 97%, at least 98%, at least 99%, or 100% sequence identity to a naturally occurring recombinase. In some embodiments, a recombinase polypeptide comprises a serine recombinase, e.g., a serine integrase. In some embodiments, a serine recombinase, e.g., a serine integrase, comprises one or more (e.g., all) of a recombinase domain, a catalytic domain, or a zinc ribbon domain. In some instances, a recombinase polypeptide has one or more functional features of a naturally occurring recombinase. In some embodiments, a recombinase polypeptide comprises a tyrosine recombinase, e.g., a tyrosine integrase.

In accordance with the present invention, an improved feature of the one-vector system is to incorporate the corresponding target site sequence that is recognizable by the genome modifying enzyme in the same vector (e.g., a recognition sequence specific to a recombinase). As used herein, a target site sequence, e.g., a recognition sequence, generally refers to a nucleic acid (e.g., DNA) sequence that is recognized (e.g., capable of being bound by) by a genome modifying enzyme. In general, a genome modifying enzyme recognizes and binds to its target site sequence before catalyzing nucleic acid modification. The target site sequence may be positioned in the modification site (the site into which a nucleic acid is to be modified) and a sequence adjacent a nucleic acid of interest to be introduced into the modification site.

As non-limiting examples, the target site sequence comprises about 10-2,000nucleotides (nts), 10-1,500 nts, 10-1,000 nts, 10-500 nts, 10-200 nts, 20-2,000 nts, 20-1,500 nts, 20-1,000 nts, 20-800 nts, 20-500 nts, 20-200 nts, 20-100 nts, 50-2,000 nts, 50-1,500 nts, 50-1,000 nts, 50-500 nts, 50-200 nts, 100-2,000 nts, 100-1,500 nts, 100-800 nts, 100-500 nts, 200-1,500 nts, 200-800 nts, 500-2,000 nts, 500-1,000 nts or 500-800 nts. For example, the target site sequence comprises about 10 nts, about 15 nts, about 20 nts, about 25 nts, about 30 nts, about 40 nts, about 50 nts, about 60 nts, about 70 nts, about 80 nts, about 90 nts, about 100 nts, about 150 nts, about 200 nts, about 250 nts, about 300 nts, about 400 nts, about 500 nts, about 600 nts, about 700 nts, about 800 nts, about 1,000 nts, about 1,200 nt, about 1,500 nts, about 2,000 nts, or more than 2,000 nts.

The target site sequence may be a native target sequence that is recognizable by a genome modifying enzyme, or alternatively an artificial designed sequence that is recognizable by a genome modifying enzyme. The target site or recognition sequence for a specific enzyme is a naturally occurring nucleic acid sequence in a genome. The enzyme or enzyme polypeptide recognizes and binds to the target site sequence (e.g., recognition sequence) in a nucleic acid molecule to exercise its enzymatic functions.

In some embodiments, the target site sequence comprises a nucleic acid sequence having at least 70%, at least 75%, at least 80%, at least 85%, at least 90%, at least 95%, at least 96%, at least 97%, at least 98%, at least 99%, or 100% sequence identity to a naturally occurring target site sequence of an enzyme.

The target site sequence may be inserted into the vector at any position. In some embodiments, the target site sequence locates at the 3′ end of the polynucleotide encoding the enzyme, or the 5 end of the polynucleotide encoding the enzyme.

Taking serine recombinase as an illustrative example, the integrated vector comprising a serine recombinase (or a serine recombinase polypeptide) comprises a first recognition sequence of the recombinase, e.g., an attP sequence or a pseudo-attP sequence. In other examples, the integrated vector comprises a serine recombinase (or a serine recombinase polypeptide) may comprise a second recognition sequence of the recombinase, e.g., an attB sequence or a pseudo-attB sequence. In some examples, the integrated vector comprises an attP sequence or an attB sequence. In yet other examples, the integrated vector comprises a pseudo-attP sequence or a pseudo-attB sequence. As used herein, the term “att” refers to the attachment site. The attP sequence refers to the attachment site sequence in the phage genome. The attB site sequence refers to the attachment site sequence in the host (e.g., bacterial) genome. A serine recombinase directs site specific integration between the attP and attB sequences recognized by the serine recombinase.

The recognition sequences for LSRs are typically arranged as follows. An AttB comprises a first DNA sequence attB 5′, a core region, and a second DNA sequence attB3′,in the relative order from 5′ to 3′: attB5′-core region-attB3′. An AttP comprises a first DNA sequence attP5′, a core region, and a second DNA sequence attP3′, in the relative order from 5′ to 3′: attP5′-core region-attP3′. In some embodiments, the attB 5′ and attB 3′ are parapalindromic (e.g., one sequence is a palindrome relative to the other sequence or has at least 20%, 30%, 40%, 50%, 60%, 70%, 75%, 80%, 85%, 90%, 95%, 96%, 97%, 98%, or 99% sequence identity to a palindrome relative to the other sequence). In some embodiments, the attP5′ and attP3′ recognition sequences are parapalindromic (e.g., one sequence is a palindrome relative to the other sequence or has at least 20%, 30%, 40%, 50%, 60%, 70%, 75%, 80%, 85%, 90%, 95%, 96%, 97%, 98%, or 99% sequence identity to a palindrome relative to the other sequence). In some embodiments, the attB 5′ and attB 3′ recognition sequences are parapalindromic to each other. In other embodiments, the attP5′ and attP3′ recognition sequences are parapalindromic to each other. In some embodiments, the attB 5′ and attB3′, and the attP5′ and attP3′ sequences are similar but not necessarily the same number of nucleotides.

In some embodiments, the recognition sequence specific to a large serine recombinase (e.g., attP and attB sequences) may comprise about 15-150 nts, or 15-120 nts, or 15-100 nts, or 15-75 nts, or 20-150 nts, or 20-120 nts, or 20-100 nts, or 20-75 nts, or 30-150 nts, or 30-120 nts, or 30-100 nts, or 30-75 nts, or 50-150 nts, or 50-120 nts, or 50-100 nts, or 50-75 nts. In some embodiments, the recognition sequence comprises about 30-75 nts. In one embodiment, the attP sequence is about 30-75 nts in length, e.g., 30 nts, 31 nts, 32 nts, 33 nts, 34 nts, 35 nts, 36 nts, 37 nts, 38 nts, 39 nts, 40 nts, 41 nts, 42 nts, 43 nts, 44 nts, 45 nts, 46 nts, 47 nts, 48 nts, 49 nts, 50 nts, 51 nts, 52 nts, 53 nts, 54 nts, 55 nts, 56 nts, 57 nts, 58 nts, 59 nts, 60 nts, 61 nts, 62 nts, 63 nts, 64 nts, 65 nts, 66 nts, 67 nts, 68 nts, 69 nts, 70 nts, 71 nts, 72 nts, 73 nts, 74 nts, or 75 nts in length. In one embodiment, the attB sequence is about 30-75 nts in length, e.g., 30 nts, 31 nts, 32 nts, 33 nts, 34 nts, 35 nts, 36 nts, 37 nts, 38 nts, 39 nts, 40 nts, 41 nts, 42 nts, 43 nts, 44 nts, 45 nts, 46 nts, 47 nts, 48 nts, 49 nts, 50 nts, 51 nts, 52 nts, 53 nts, 54 nts, 55 nts, 56 nts, 57 nts, 58 nts, 59 nts, 60 nts, 61 nts, 62 nts, 63 nts, 64 nts, 65 nts, 66 nts, 67 nts, 68 nts, 69 nts, 70 nts, 71 nts, 72 nts, 73 nts, 74 nts, or 75 nts in length.

A pseudo-recognition sequence may be incorporated in the vector. In the context of recombinases, e.g., serine recombinases, recognition sequences exist in the genomes of a variety of organisms, where the recognition sequence does not necessarily have a nucleotide sequence identical to the wild-type recognition sequences (for a given recombinase); but such native recognition sequences are nonetheless sufficient to promote recombination meditated by the recombinase. Such recognition sequences are among those referred to herein as “pseudo recognition sequences.” A “pseudo-recognition sequence” is a DNA sequence comprising a recognition sequence that is recognized (e.g., capable of being bound by) by a recombinase enzyme, where the recognition sequence: differs in one or more nucleotides from the corresponding wild-type recombinase recognition sequence, and/or is present as an endogenous sequence in a genome that differs from the sequence of a genome where the wild-type recognition sequence for the recombinase resides. A pseudo attP sequence may be a native genomic sequences, e.g., found in human genome, similar to the bona fide attP sites that could be recognized by a LSR. A pseudo attP sequence could also include mutated attP sites from the natural attP sequence recognized by a LSR.

In some embodiments, for a given recombinase, a pseudo-recognition sequence is functionally equivalent to a wild-type recombination sequence, occurs in an organism other than that in which the recombinase is found in nature, and may have sequence variation relative to the wild type recognition sequences. “Pseudo attP site” or “pseudo attB site” refer to pseudo-recognition sequences that are similar to the recognition sequences for wild-type phage (attP) or bacterial (attB) attachment site sequences, respectively, e.g., for phage integrase enzymes, such as the phage PhiC31. In some embodiments, the attP or pseudo attP site is present in the genome of a host cell, while the attB or pseudo attB site is present on a targeting vector in a system described herein. In some embodiments, the attB or pseudo attB site is present in the genome of a host cell, while the attP or pseudo attP site is present on a targeting vector in a system described herein. “Pseudo att site” is a more general term that can refer to either a pseudo attP site or a pseudo attB site. An att site or pseudo att site may be present on a linear or a circular nucleic acid molecule. Identification of pseudo-recognition sequences can be accomplished, for example, by using sequence alignment and analysis, where the query sequence is the recognition sequence of interest (for example an attB and/or attP of a phage/bacterial system). For example: if a genomic recognition sequence is identified using an attB query sequence, then it is said to be a pseudo-attB site; if a genomic recognition sequence is identified using an attP query sequence, then it is said to be a pseudo-attP site. In some embodiments, the pseudo-recognition sequences share high sequence similarity with wild-type recognition sequences recognized by (e.g., capable of binding to) the recombinase (e.g., one or more of the aE helix, recombinase domain, the linker domain, and/or the zinc ribbon domain as described in Li H et al., 2018, J Mol Biol, 430 (21): 4401-4418, which is incorporated herein by reference in its entirety). In some embodiments, pseudo-recognition sequences are more strongly bound or acted upon by a recombinases than the wild-type recognition sequence of the recombinase. A pseudo-recognition sequence may also be referred to as a “pseudosite.” In some embodiments, a pseudosite may be quite divergent from a parental sequence, e.g., as described in Thyagarajan et al., Mol Cell Biol 21(12):3926-3934 (2001). In some embodiments, a pseudosite as used herein may be less than 70%, e.g., less than 70%, 60%, 50%, 40%, or less than 30% identical to a native recognition sequence. In some embodiments, a pseudosite as used herein may be more than 20%, e.g., more than 20%, 30%, 40%, 50%, 60%, or more than 70% identical to a native recognition sequence.

As non-limiting examples, a pseudo-recognition sequence (e.g., a human DNA recognition sequence) is incorporated into the vector comprising a serine recombinase, where the pseudo-recognition sequence is derived from a position in or near (e.g., within 1, 2, 3, 4, 5, 6, 7, 8, 9, 10, 15, 20, 25, 30, 35, 40, 45, 50, 55, 60, 65, 70, 75, 80, 90, 100, 150, 200, 300, 400, 500, 600, 700, 800, 900, 1000, 2000, 3000, 4000, 5000, or 10,000 nucleotides of) a genomic safe harbor site. A genomic safe harbor (GSH) site is a site in a host genome that is able to accommodate the integration of new genetic material, e.g., such that the inserted genetic element does not cause significant alterations of the host genome posing a risk to the host cell or organism. A GSH site generally meets 1, 2, 3, 4, 5, 6, 7, 8 or 9 of the following criteria: (i) is located >300 kb from a cancer-related gene; (ii) is >300 kb from a miRNA/other functional small RNA; (iii) is >50 kb from a 5′ gene end; (iv) is >50 kb from a replication origin; (v) is >50 kb away from any ultraconserved element; (vi) has low transcriptional activity (i.e. no mRNA +/−25 kb); (vii) is not in a copy number variable region; (viii) is in open chromatin; and/or (ix) is unique, with 1 copy in the human genome. Examples of GSH sites in the human genome that meet some or all of these criteria include (i) the adeno-associated vims site 1 (AAVS1), a naturally occurring site of integration of AAV vims on chromosome 19; (ii) the chemokine (C-C motif) receptor 5 (CCR5) gene, a chemokine receptor gene known as an HIV-1 coreceptor; (iii) the human ortholog of the mouse Rosa26 locus; (iv) the rDNA locus. Additional GSH sites are known and described, e.g., in Pellenz et al. epub Aug. 20, 2018 (https://doi.org/10.1101/396390).

In some exemplary embodiments, the recognition sequence of a LSR in the present vector is derived from a sequence within about 200-400 bps downstream of the coding sequence of the LSP in the phage genome. In some examples, the recognition sequence of a LSR in the vector is derived from a sequence within about 200-300 bps downstream of the coding sequence of the LSP in the phage genome. In some examples, the recognition sequence of a LSR in the vector is derived from a sequence within about 100-400 bps downstream of the coding sequence of the LSP in the phage genome. In some examples, the recognition sequence of a LSR in the vector is derived from a sequence within about 150-300 bps downstream of the coding sequence of the LSP in the phage genome. In some examples, the recognition sequence of a LSR in the vector is derived from a sequence within about 150-350 bps downstream of the coding sequence of the LSP in the phage genome. In some examples, the recognition sequence of a LSR in the vector comprises a sequence within about 200 bps downstream of the coding sequence of the LSP in the phage genome. In some examples, the recognition sequence of a LSR in the vector comprises a sequence within about 250 bps downstream of the coding sequence of the LSP in the phage genome. In some examples, the recognition sequence of a LSR in the vector comprises a sequence within about 300 bps downstream of the coding sequence of the LSP in the phage genome.

In other embodiments, the recognition sequence of a LSR in the present vector is derived from a sequence within about 200-400 bps upstream of the coding sequence of the LSP in the phage genome. In some examples, the recognition sequence of a LSR in the vector is derived from a sequence within about 200-300 bps upstream of the coding sequence of the LSP in the phage genome. In some examples, the recognition sequence of a LSR in the vector is derived from a sequence within about 100-400 bps upstream of the coding sequence of the LSP in the phage genome. In some examples, the recognition sequence of a LSR in the vector is derived from a sequence within about 150-400 bps upstream of the coding sequence of the LSP in the phage genome. In some examples, the recognition sequence of a LSR in the vector is derived from a sequence within about 1500-350 bps upstream of the coding sequence of the LSP in the phage genome. In some examples, the recognition sequence of a LSR in the vector comprises a sequence within about 200 bps upstream of the coding sequence of the LSP in the phage genome. In some examples, the recognition sequence of a LSR in the vector comprises a sequence within about 250 bps upstream of the coding sequence of the LSP in the phage genome. In some examples, the recognition sequence of a LSR in the vector comprises a sequence within about 300 bps upstream of the coding sequence of the LSP in the phage genome.

Taking transposases as another representative example, the integrated vector may comprise a polynucleotide encoding a transposase, a nucleic acid sequence for the terminal inverted repeats (TIRs) that are specific to the transposase and a unique identifier that correlates to the transposase. A transposase recognizes and interacts with its binding sites in the terminal inverted repeats (TIRs) that define the boundaries of the transposon to initiate the transposition process.

In some embodiments, the target site sequence is a hybrid target site sequence (e.g., a hybrid recognition sequence). As used herein, the term “hybrid target site sequence” refers to a sequence constructed from portions of a plurality of targeting sequences, e.g., wild type and/or pseudo-targeting sequences.

In some embodiments, more than one target site sequence (e.g., recognition sequences) that are recognizable by the same enzyme are included in the vector, for example, two, three or more sequences are included. In some embodiments, the multiple target site sequences comprise the same sequence. In other embodiments, the multiple target site sequences comprise different sequences.

In some embodiments, the target site sequence that is specific to a genome modifying enzyme comprises a continuous sequence; in other embodiments, the target site sequence is separated, e.g., two fragments and three fragments.

In some embodiments, the integrated vector comprises at least one unique identifier that correlates to the genome modifying enzyme in the vector.

In accordance with the present invention, the unique identifier is a nucleic acid-based identifier. The unique identifier is a barcode, e.g., a DNA barcode and a RNA barcode. The barcode sequence comprises a random sequence but the sequence does not affect expression of the encoded enzyme nor its genomic modification function (e.g., binding to its target site sequence).

In one non-limiting example, the nucleic acid identifier comprises a short nucleotide sequence. In some embodiments, the identifier comprises about 4-30 nucleotides. In some embodiments, the identifier comprises about 5-25 nucleotides. In some embodiments, the identifier comprises about 6-25 nucleotides. In some embodiments, the identifier comprises about 6-20 nucleotides. In some embodiments, the identifier comprises about 6-15 nucleotides. In some embodiments, the identifier comprises about 6-10 nucleotides. In some embodiments, the identifier is about 4, 5, 6, 7, 8, 9, 10, 11, 12, 13, 14, 15, 16, 17, 18, 19, 20, 21, 22, 23, 24, 25, 26, 27, 28, 29, or 30 nucleotides in length or longer. In some embodiments, the length of a identifier is about 4, 5, 6, 7, 8, 9, 10, 11, 12, 13, 14, 15, 16, 17, 18, 19, 20, 21, 22, 23, 24, 25, 26, 27, 28, 29, or 30 nucleotides in length or shorter. In some embodiments, the identifier is at least 4 nucleotides in length. In some embodiments, the identifier is at least 5 nucleotides in length. In some embodiments, the identifier is at least 6 nucleotides in length. In some embodiments, the identifier is at least 7 nucleotides in length. In some embodiments, the identifier is at least 8 nucleotides in length. In some embodiments, the identifier is at least 9 nucleotides in length. In some embodiments, the identifier is at least 10 nucleotides in length. In some embodiments, the identifier is at least 11 nucleotides in length. In some embodiments, the identifier is at least 12 nucleotides in length. In some embodiments, the identifier is at least 13 nucleotides in length. In some embodiments, the identifier is at least 14 nucleotides in length. In some embodiments, the identifier is at least 15 nucleotides in length. In some embodiments, the identifier is at least 16 nucleotides in length. In some embodiments, the identifier is at least 17 nucleotides in length. In some embodiments, the identifier is at least 18 nucleotides in length. In some embodiments, the identifier is at least 19 nucleotides in length. In some embodiments, the identifier is at least 20 nucleotides in length. In some embodiments, the identifier is at least 21 nucleotides in length. In some embodiments, the identifier is at least 22 nucleotides in length. In some embodiments, the identifier is at least 23 nucleotides in length. In some embodiments, the identifier is at least 24 nucleotides in length. In some embodiments, the identifier is at least 25 nucleotides in length. In some embodiments, the identifier is at least 26 nucleotides in length. In some embodiments, the identifier is at least 27 nucleotides in length. In some embodiments, the identifier is at least 28 nucleotides in length. In some embodiments, the identifier is at least 29 nucleotides in length. In some embodiments, the identifier is at least 30 nucleotides in length.

In one embodiment, the nucleic acid identifier comprises 20 nucleotides.

In some embodiments, the identifier comprises unmodified nucleotides. In some embodiments, the identifier comprises modified nucleotides. In some embodiments, the identifier comprises a combination of unmodified and modified nucleotides.

In some embodiments, the nucleic acid identifier may inserted at the 5′ end of the polynucleotide encoding the genome modifying enzyme, at the 3′ end of the polynucleotide encoding the genome modifying enzyme, or at the 3′ end of the target site sequence, or at any other locations within the vector depending on the vector design.

The integrated vector comprises other components such as additional polynucleotides and regulatory sequences.

In some embodiments, the integrated vector comprises one or more reporter genes such as fluorescent proteins. The fluorescent proteins can be GFP, YFP, RFP, BFP, CFP, mEGFP, EGFP, mApple, mCherry, tdTomato, mTurquoise, mTagBFP, mKO2, mKate2, efasGFP, aeurGFP, Skylan-S, PlamGFP, eechGFP1, pcDronpa, dVFP, RRvT, eEosEM, ccalYFP1, bfloGFPa1, LanYFP, dLanYFP, AausFP1, h2-3,vsfGFP-0, StayGold, mNeonGreen, Kaede, mClove3, Clover, VFP, pcDronpa2, moxneonGreen, tdimer2, Dronpa, YPet, Skylan-NS, ffDronpa, fusionRed-MQV, eechGFP2, gfasGFP, Gamillus, sarGFP, vsfGFP-9, eEos3.1, mGreenLantern, pmeaGFP1, mVFP, pmimGFP1, pmimGFP2, eYGFP, mEos4a, pcDronpa2, pdae 1GFP, pdaeGFP2, mScarlet, mCitrine, ccalGFp3, phiYFP, GRvT, SYFP2, Citrine2, mGold, mVFP1, Gamillus0.4, SHardonnay, aacuGFP2, m Venus, pcStar, mEos4b, mKOk, fabdGFP, mGeos-C, plobRFP, rsKame, TurboRFP, afraGFP, stylGFP, azaleaB5, super-tagRFP, phiYFPv, Folding Reporter GFP, Citrine, dendFP, PSmOrange, anobGFP, mRuby3, RFP611, Topaz, SEYFP, mScarlet-1, mWasabi, iq-mVenus, meffRFP, d2EosFP, eqFP578, EYFP-Q69K, mTFP1, ccalRFP1, eechGFP3, cgreGFP, Superfolder GFP, CpYGFP, meffGFP, mEos3.2, pporGFP, muGFP, Venus, mGeos-E, Gamillus0.2, mEosFP-M159A (green), pporRFP, pcDronpa (Red), d1EosFP (green), moxGFP, oxGFP, EosFP, mCherry-XL, amilFP513, KO, DsRed, mEYFP, mOrange, sg11, sg12, mc1, mc2, mc3, mc5, mc6, PH-tdGFP, psupFP, ptilGFP, Q80R, RCaMP, R-FlincA, rfloGFP, rfloGFP2, rfloRFP, RFP618, roGFP1, roGFP1-R1, roGFP1-R8, roGFP2, RpBphP1, RpBphP2, RpBphP6, rrenGFP, rrGFP, rsCherryRev1.4, RSGFP1, RSGFP2, RSGFP3, RSGFP4, RSGFP5, RSGFP6, RSGFP7, Rtms5, SAASoti (green), SAASoti (red), scleFP1, scleFP2, scubGFP1, scubGFP2, secBFP2, sfCherry, sfCherry2, sfCherry3C, SH3, ShG24, ShyRFP, spGFP 11, spGFP1-10, spisCP, stylCP, Superfolder BFP, Superfolder CFP, Superfolder mTurquoise2, Superfolder mTurquoise2 ox, Superfolder YFP, SuperNova2, sympFP, TeAPCα, tpapaya0.01, TripartiteGFP, Trp-less GFP, TurboGFP-V197L, V127T SAASoti (green), V127T SAASoti (red), vsGFP, Xpa, yEGFP, YFP3, zoan2RFP, zRFP and variants thereof.

In one exemplary embodiment, the reporter is GFP or a variant thereof.

In some embodiments, the integrated vector comprises one or more selective markers. Commonly, genes that confer resistance to various antibiotics are used as selective markers in vectors, e.g., kanamycin, Hygromycin B, tetracycline, neomycin and puromycin. In some embodiments, the antibiotic resistance marker is a puromycin resistance marker.

In some embodiments, the integrated vector comprises at least one promoter. The present invention may encompass the use of constitutive promoters which direct expression of the nucleic acid molecules in many types of host cells and those which direct expression only in certain host cells (e.g., tissue-specific regulatory sequences) or in response to specific events or exogenous factors (e.g., by temperature, nutrient additive, hormone or other ligand). Suitable promoters for constitutive expression in eukaryotic systems include viral promoters, such as SV40 promoter, the cytomegalovirus (CMV) immediate early promoter or enhancer, the adenovirus early and late promoters, the thymidine kinase (TK) promoter of herpes simplex virus (HSV)-1 and retroviral long-terminal repeats (e.g., MoMuLV and Rous sarcoma virus (RSV) LTRs) as well as cellular promoters such as the phosphoglycero kinase (PGK) promoter.

Inducible promoters may be used. Inducible promoters are regulated by exogenously supplied compounds, and include, without limitation, the zinc-inducible metallothionein (MT) promoter, the dexamethasone (Dex)-inducible mouse mammary tumor virus (MMTV) promoter, the T7 polymerase promoter system, the ecdysone insect promoter, the tetracycline-repressible promoter, the tetracycline-inducible promoter, the RU486-inducible promoter, the rapamycin-inducible promoter and the lac, TRP, and TAC promoters from E. coli.

In one exemplary embodiment, the promoter is a CMV promoter.

In certain embodiments, a specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. In some embodiments, the vector may generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript.

It is also understandable to those skilled in the art that one or more linker sequences may be used to operably link one or more polynucleotide sequences within the integrated vector. In some embodiments, a cleavable linker may be used. In other embodiments, the linker is non-cleavable. For example, a 2A peptide linker (e.g., E2A, T2A and P2A) is inserted to separate the reporter gene (e.g., GFP) from the rest of the sequence of the vector.

Those skilled in the art will appreciate that the regulatory elements controlling the expression of the nucleic acid molecules comprised in the vector of the invention may further comprise additional elements for proper initiation, regulation and/or termination of transcription (e.g. poly(A) transcription termination sequences), mRNA transport (e.g., nuclear localization signal sequences), processing (e.g., splicing signals), stability (e.g., introns and non-coding 5′ and 3′ sequences), and translation (e.g. tripartite leader sequences, ribosome binding sites, Shine-Dalgamo sequences, etc.) into the host cell.

As will be appreciated by one skilled in the art, methods of designing and constructing nucleic acid vector constructs are routine in the art. Generally, recombinant methods may be used. Methods of designing, preparing, evaluating, purifying and manipulating nucleic acid compositions are described in e.g., Green and Sambrook (Eds.), Molecular Cloning: A Laboratory Manual (Fourth Edition), Cold Spring Harbor Laboratory Press (2012).

The system may comprise codon optimized sequences. In some embodiments, only the e.g., the human codon optimized enzyme encoding sequences are included in the system described herein. In some embodiments, the full sequence of the system is codon optimized. In some aspects, the nucleic acid sequence of the system comprises at least one chemically modified nucleotide. In other aspects, the nucleic acid sequence of the system does not comprise modified nucleotides.

### Vector Types

The integrated vector can be any vector that is capable of expressing the polynucleotide parts of the integrated vector in a host cell or subject. According to the present disclosure, the integrated vector comprising a genome modifying enzyme and its target site sequence is a heterogeneous vector, distinct from a naturally occurring organism, e.g., a bacteriophage. In one aspect, the vector is a non-viral based vector. In another aspect, the vector is a viral based vector.

The vector may be extrachromosomal (e.g., episome) or integrating (for being incorporated into the host chromosomes), autonomously replicating or not, multi or low copy, double-stranded or single-stranded, naked or complexed with other molecules (e.g., vectors complexed with lipids or polymers to form particulate structures such as liposomes, lipoplexes or nanoparticles, vectors packaged in a viral capsid, and vectors immobilized onto solid phase particles, etc.).

In some embodiments, the vector is a non-viral vector (e.g., a plasmid, cosmid, artificial chromosome and the like).

In some embodiments, the vector of the present invention is an adenoviral vector. It can be derived from any human or animal adenovirus. Any serotype and subgroup can be employed in the context of the invention. One may cite more particularly subgroup A (e.g. serotypes 12, 18, and 31), subgroup B (e.g. serotypes 3, 7, 11, 14, 16, 21, 34, and 35), subgroup C (e.g. serotypes 1, 2, 5, and 6), subgroup D (e.g. serotypes 8, 9, 10, 13, 15, 17, 19, 20, 22-30, 32, 33, 36-39, and 42-47), subgroup E (serotype 4), and subgroup F (serotypes 40 and 41). Particularly preferred are human adenoviruses 2 (Ad2), 5 (Ad5), 6 (Ad6), 11 (Ad11), 24 (Ad24) and 35 (Ad35). Such adenoviruses are available from the American Type Culture Collection (ATCC, Rockville, Md.) and have been the subject of numerous publications describing their sequence, organization and methods of producing, allowing the artisan to apply them.

Adenoviral vectors have approximately 8 to 30 kilobase (kb) capacity, can be used as nucleic acid delivery tools with 100% efficiency to a wide selection of cell types including dividing and non-dividing cells, primary cells, or cell lines. In some embodiments, recombinant adenoviral vectors may be generated for use in the present systems. In some examples, recombinant adenoviral vectors have ability to infect most mammalian cell types (both dividing and non-dividing cells) and allow high and stable expression of the transferred genes.

In some embodiments, the vector used in the present system is an adeno-associated viral (AAV) vector. AAV is a small non-enveloped parvovirus with a single-stranded genome of about 5 kb that is naturally non-pathogenic and replication defective. AAV vectors contain no viral coding sequences, and commonly used to deliver nucleic acid molecules into cells and organisms. An AAV-based vector may be made possible by the isolation of several naturally occurring AAV serotypes and over 100 AAV variants from different animal species. In some aspects, the AAV vector can be any serotype such as AAV1, AAV2, AAV3, AAV4, AAV5, AAV6, AAV7, AAV8, AAV9, AAV10,AAV11, AAV12, and variants thereof. In other aspects, the vector is a hybrid AAV vector derived from different AAV serotypes. A chimeric AAV vector refers to the vector containing capsid proteins that have been modified by domain or amino acid swapping between different serotypes. Strategies for the generation of chimeric virions primarily involve the marker rescue approach or mutagenesis of AAV virions to swap surface domains ranging from single to multiple amino acid residues.

Exemplary AAV vector may be selected from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9,45, AAV9.47, AAV9.61, AAV9.68, AAV9.84, AAV9,9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAVI-8/rh.49, AAV2-15/th.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rl 1,64, AAV4-9/rh.54, AAV4-19/rh.55, AAV5-3/rh.57, AAV5-22/rh.58, AAV7.3/hu.7, AAV16.8/hu. 10, AAV16.12/hu.1 1, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu. 15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5RI, AAVCy.5R2, AAVCy.5R3, AAVCy.5R4, AAVcy.6, AAVhu.l, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AA Vhu.25, AA Vhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu. 14/9, AA Vhu.t 19, AAVrh.2, AAVrh.2R, AAVrh.8, AAVrh.8R, AAVrh.1O, AAVrh.12, AAVrh.13, AAVrh.BR, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64Rl, AAVrh.64R2, AAVrh.67, AAVrh.73, and/or AAVrh.74.

In some embodiments, the vector of the present system is a lentiviral vector, such as a recombinant lentiviral vector. Lentiviral vectors (LV) are based on the single-stranded RNA lentiviruses, which are a subclass of retrovirus. A recombinant lentiviral vector can be a SIV or HIV vector carrying polynucleotide of any genome modifying enzyme, its recognition site sequence and a unique identifier that correlates to the genome modifying enzyme. The recombinant lentiviral vector can be a recombinant simian immunodeficiency virus (SIV) based vector or a recombinant human immunodeficiency virus (HIV) based vector, or equine infectious anemia virus (EIAV) based vector, or Feline Immunodeficiency Virus (FIV) based vector. The human immunodeficiency virus includes all HIV strains and sub-types thereof. HIV includes two types of viral strains: HIV-1 and HIV-2. HIV-1 is divided into M, O, N sub-type groups. M sub-type group includes sub-types A, A2, B, C, D, E, F1, F2, G, H, J, K, while O and N sub-types is scarcely seen. HIV-2 includes A, B, C, D, E, F, G sub-types.

In some embodiments, the vector of the present system is a retroviral vector, such as aculovirus expression vectors (BEVs), and herpes simplex virus type-1 (HSV) vectors to benefit from their large payload capacity (up to 36 kb, 50 kb, and 130 kb, respectively).

In some embodiments, the vector of the present system is a rabies viral vector, for example, a recombinant rabies viral vector, and a pseudotyped rabies viral vector. A recombinant rabies viral vector may lack a G gene encoding for a rabies virus glycoprotein or a functional variant thereof. In some cases, a recombinant rabies viral vector lacks an L gene encoding for a rabies virus polymerase or a functional variant thereof. In some embodiments, a recombinant rabies viral vector may further lack a M gene encoding for a rabies virus matrix protein or a functional variant thereof; a P gene encoding for a rabies virus phosphoprotein or a functional variant thereof; and/or an N gene encoding for a rabies virus nucleoprotein or a functional variant thereof. In other embodiments, the rabies viral vector is pseudotyped comprising an altered G-protein, e.g., a G-protein derived from other viruses.

In some embodiments, the integrated system may be in a format of in vitro-transcribed messenger RNA (mRNA). For example, a mRNA polynucleotide comprises a first polynucleotide encoding a genome modifying enzyme, a second polynucleotide of a target site sequence that is recognizable by the genome modifying enzyme and a unique RNA barcode, and polynucleotides encoding other components of the system, e.g., mRNA encoding a reporter gene (e.g., GFP). The mRNA molecule may be formulated in a lipid-based vehicle for cell transfection such as lipid nanoparticles and liposomes.

In some embodiments, the polynucleotide is codon optimized. Codon optimization is the process of modifying the coding region of a gene to more closely align the codon usage of a gene of interest with the codon usage frequency or codon bias of the target cell or organism, while retaining the same amino acid coding sequence. In some instances, codon optimization may improve translation efficiency. Numerous codon usage tables are publicly available and may be found, for example at https://www.genscript.com/tools/codon-frequency-tablem or https://www.kazusa.or.jp/codon/. See also Athey et al., A new and updated resource for codon usage tables, BMC Bioinformatics. 2017; 18:391 (2017).

In accordance with the present invention, the integrated vector can be made using any known recombinant nucleic acid technologies, such as recombinant technology, PCR and in vitro synthesis, etc.

### One Vector Systems and Assays

In some embodiments, the one vector system of the present invention comprises a plurality of integrated vectors, each of which comprises a polynucleotide encoding a unique genome modifying enzyme, a target site sequence that is recognizable by the unique genome modifying enzyme in the same vector, and a unique identifier that correlates to the unique genome modifying enzyme in the vector.

In some embodiments, the one vector system may comprises a plurality of integrated vectors encoding 50-20,000, 100 to10,000, 200 to 10,000, 300 to 10,000, 400 to 10,000, 500 to10,000, or 1,000 to 10,000, 2,000 to 10,000, or 5,000 to 10,000 unique enzymes, for example, 50 or more, 100 or more, 200 or more, 300 or more, 400 or more, 500 or more, 600 or more, 1,000 or more, 2,000 or more, 3,000 or more, 5,000 or more, 6,000 or more, 7,000 or more, 8,000 or more, 9,000 or more, 10, 000 or more, 15,000 or more, 20,000 or more, or more than 20,000 unique genome modifying enzymes.

In some embodiments, the one vector system comprises a plurality of integrated vectors as described herein, which are pooled together to form a library of enzymes or enzyme polypeptides.

The one vector system of the present invention is used to identify a genome modifying enzyme. In some embodiments, a method for identifying a genome modifying enzyme using the one-vector system is provided. The process for identify a genome modifying enzyme for genome modification comprises: i) transfecting into cells a plurality of vectors, each of which comprises a polynucleotide encoding a unique genome modifying enzyme, a target site sequence that is recognizable by the unique genome modifying enzyme in the vector and a unique identifier that correlates to the genome modifying enzyme in the vector; ii) detecting genome modification activities in the transfected cells; and iii) identifying the genome modifying enzyme by identifying the unique identifier in the transfected cells in which the genome modification activities are detected.

Various cells can be employed to the present methods. In some embodiments, the cells are mammalian cells. In some embodiments, the cells are human cells. In some aspects, the cells are actively dividing. In certain aspects transfection involves transfection of a cell line or a hybrid cell type. Examples of cells include A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293 FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NSO, Panc-1, PC12, PC-3, Peripheral blood cells, Plasma cells, Primary Fibroblasts, RBL, Renca, RLE, SF21, SF9, SH-SY5Y, SK-MES-1, SK-N-SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, and hematopoietic stem cells.

Any transfection agents and methods known in the art can be used for the present method, such as chemical transfection methods and physical transfection. Chemical transfection is a popular technique due to the ease, cost, and wide variety of transfection reagents available. A chemical transfection includes plating cells at subconfluency, preparing the transfection reagent/DNA complex immediately before transfection, and then assessing construct expression. Cell culture medium is refreshed a day after plating cells and then a few hours after transfection. Optimization of the transfection protocol may be undertaken to ensure high transfection efficiency and that the method is not toxic to the cells being transfected. Exemplary chemical transfection methods and agents include liposomes, cationic polymers (e.g., dextran, polybrene polyethylenimine and dendrimers), calcium phosphate co-precipitation and other nonliposomal reagents. Physical transfection involves electroporation, microinjection, biolistic particle delivery, and cell injection (e.g., gene guns).

In some embodiments, the transfection method used is a chemical based transfection method, e.g., calcium phosphate, dendrimers, lipofection, and cationic polymers such as DEAE-dextran or polyethylenimine.

In other embodiments, the transfection method used is a non-chemical based transfection method, e.g., cell squeezing, sonoporation, optical transfection, impalefection, hydrodynamic delivery, magnetofections (i.e., magnet-assisted transfection), and particle bombardment.

In certain embodiments, the transfection method used is electroporation. In a further embodiment, the electroporation method is flow electroporation.

Any of the disclosed methods may include a step employing limiting dilution of the transfected cells to obtain single cell colonies and/or enrichment of cells with positive genome modification activity. For example, the method may further include a step comprising expanding a clonal isolated and selected cell to produce clonal cells with a particular genomic DNA sequence modification.

The cell culture may include any additional ingredients known to those of ordinary skill in the art, as would be readily selected by those of ordinary skill in the art based on the type of cell that is cultured. For example, the cells may be cultured in sodium butyrate or comparable salt. In some embodiments, the cells are cultured in serum-free media.

In certain embodiments, a further step may be employed comprising expanding a clonal isolated and selected or screened cell to produce clonal cells having a genomic DNA sequence modification.

In some embodiments, the genome modification events are detected in the transfected cells. As non-limiting examples, the modifications are detected at least about 1 day, 2 days, 3 days, 4 days, 5 days, 6 days, 1 week, or 2 weeks or more after transfection. In one example, the modifications are detected one week after transfection.

A genomic modification event may be detected using any methods known in the art that is suitable for detecting a specific modification. In some embodiments, the genomic modification events are detected using next generation sequencing (NGS). Next-generation sequencing (NGS) is a massively parallel sequencing technology that offers ultra-high throughput, scalability, and speed. The rapid development of the next-generation sequencing technologies has given unprecedented power to solve problems in multiple fields of molecular biology, resulting in many discoveries and new insights. With the emergence of new library preparation methods, computing pipelines for processing the huge volumes of sequencing data, and enhanced analysis strategies, NGS is being applied in many areas.

In some embodiments, NGS is used to detecting the genome modification mediated by a genome modifying enzyme. The sequencing method may include pyrosequencing, ion torrent sequencing, Illumina sequencing technology, Large Fragment Single Molecule Sequencing such as PacBio sequencing developed by Pacific Biosciences, and Nanopore-based DNA sequencing.

The pyrosequencing methods can be used for genome sequencing and metagenome samples because of the long read lengths (up to 600-800 nts),

The “Ion Torrent™ technology directly converts nucleotide sequence into digital information on a semiconductor chip. Ion Torrent sequencing reactions occur in millions of wells that cover a semiconductor chip containing millions of pixels that convert the chemical information into sequencing information.

A variety of protocols for Illumina sequencing including genomic sequencing, exome and targeted sequencing, metagenomics, RNA sequencing, CHIP-seq and methylome methods can be used depending on the genome modify enzyme to be screened.

PacBio sequencing, also referred to as SMRT (Singe Molecule Real Time) sequencing, enables very long fragments to be sequenced, up to 30-50 kb, or longer. An important advantage of the PacBio real time sequencing imaging and detection process is that the rate of each nucleotide addition during synthesis can be measured, termed the inter-pulse duration (IPD). Many nucleotides with base modifications, such as some adenine and cytosine methylations, change the IPD and thus can be identified as a modified base.

The nanopore based sequencing uses small diameter “holes” to pass long DNA molecules and measure differing currents as each nucleotide passes by a linked detector. Two types of nanopore systems for DNA sequencing, biological membrane systems and solid-state sensor technology can be used.

Other ancillary methods for high throughput genome sequencing, e.g., optical mapping and DNA shearing may be used to for complete large genome sequencing.

In accordance, approaches using NGS to identify and quantify genome modification activity for on-target and off-target sequence modifications provide a comprehensive analysis of nucleic acid sequence alterations in the genome.

### Genome Modifying Enzymes

In accordance with the present invention, the one vector system can be used to identify a genome modifying enzyme. Exemplary genome modifying enzymes include recombinases including serine recombinases (e.g., large and small serine recombinases) and tyrosine recombinases (e.g., small and large tyrosine recombinases), DNA transposases, retrotransposons (e.g., LTRs, LINEs and SINEs), integrases, nucleases, base editors and prime editors, etc. The genome modifying enzymes may also include engineered genome modifying enzymes, chimeric genome modifying enzymes, enzyme polypeptides, and the like.

The enzymes may be isolated from any organism. As used herein, the term “organism” encompasses microorganisms preferably having pathogenic potential and as well as higher eukaryotes. The term “microorganism” refers to fungi (e.g., yeast), bacteria, protozoa and viruses. As non-limiting examples, the enzymes are isolated or derived from bacteriophages, cyanophages, mycoviruses, archacal viruses, fungi, bacteria, animal microbiome such as human gut microbiome.

Representative examples of viruses include without limitation bacteriophages, cyanophages, mycoviruses, archaeal viruses, HIV (HIV-1 or HIV-2), human herpes viruses (e.g. HSV1 or HSV2), cytomegalovirus (CMV), Epstein Barr virus (EBV), hepatitis viruses (e.g. hepatitis A virus (HAV), HBV, HCV and hepatitis E virus), flaviviruses (e.g. Yellow Fever Virus), varicella-zoster virus (VZV), paramyxoviruses, respiratory syncytial viruses, parainfluenza viruses, measles virus, influenza viruses, and papillomaviruses, Parvoviruses, Adenoviruses, Herpesviruses, Vaccine virus, Arenaviruses, Coronaviruses, Rhinoviruses, Respiratory syncytial viruses, Influenza viruses, Picornaviruses, Paramyxovinises, Reoviruses, Retroviruses, Rhabdoviruses, or human immunodeficiency virus (HIV), Polyomaviruses, Poxviruses, Hepadnaviruses, Astroviruses, Caliciviruses, Flaviviruses, Togaviviruses, Hepeviviruses, Orthomyxoviruses, Bunyaviruses, and Filoviruses.

In some embodiments, the genome modifying enzyme is isolated or derived from a bacteriophage. Bacteriophages (or phages) refer to viruses that infect bacteria and archaea that infect and replicate in bacterial cells. Bacteriophages or phages are the most abundant organisms in the biosphere and they are a ubiquitous feature of prokaryotic existence. Bacteriophage genomes encodes enzymes that can modify host genomes (i.e., bacterial genomes) and influence their host's evolution and population dynamics. Phages are abundant in bacterial cells. Bacteriophages can profoundly influence microbial communities by functioning as vectors of horizontal gene transfer. Bacteriophages are extremely diverse in size, morphology, and genomic organization. Like all viruses, bacteriophages have replication strategies (e.g., lytic or lysogenic) to incorporate their genomic materials into host bacteria, rapidly converting the host cell resources to viral genomes to produce viral particles. During the infection process, the phage genome is either integrated into the bacterial cell chromosome or maintained as an episomal element. Under either situation, bacteriophages use a variety pf enzymes to modify the host genome. Research has attempted to use this property to identify genome modification enzymes to advance research and even for clinical uses. The most known enzymes that are used in genetic engineering are DNA polymerases encoded by bacteriophages φ29, T4 and T7. Another famous example is the CRISPR-Cas9 system which is now engineered as a tool for genetic manipulation in the lab and for gene therapy, originated as a bacterial defense mechanism against bacteriophage infection.

A genome modifying enzyme is isolated from a bacteriophage found in a bacterial cell. Representative examples of bacteria include without limitation Neisseria (e.g. N. gonorrhea and N. meningitidis); Bordetella (e.g. B. pertussis, B. parapertussis and B. bronchiseptica), Mycobacteria (e.g. M. tuberculosis, M. bovis, M. leprae, M. avium, M. paratuberculosis, M. smegmatis); Legionella (e.g. L. pneumophila); Escherichia (e.g. enterotoxic E. coli, enterohemorragic E. coli, enteropathogenic E. coli); Shigella (e.g. S. sonnei, S. dysenteriae, S. flexnerii); Salmonella (e.g. S. typhi, S. paratyphi, S. choleraesuis, S. enteritidis); Listeria (e.g. L. monocytogenes); Helicobacter (e.g. H. pylori); Pseudomonas (e.g. P. aeruginosa); Staphylococcus (e.g. S. aureus, S. epidermidis); Enterococcus (e.g. E. faecalis, E. faecium); Bacillus (e.g. B. anthracis); Corynebacterium (e.g. C. diphtheriae), Chlamydia (e.g. C. trachomatis, C. pneumoniae, C. psittaci); Xanthomonas campestris. Streptomyces coelicolo, Rhizobium sp, Agrobacterium tumefaciens; Mycobacterium tuberculosis, and C. difficile.

Representative examples of parasites include without limitation Plasmodium (e.g., P. falciparum); Toxoplasma (e.g., T. gondii); Leshmania (e.g., L. major); Pneumocystis (e.g., P. carinii); and Schisostoma (e.g., S. mansoni).

The abundance and diversity of bacteriophages can be assessed using molecular approaches (e.g., genome sequencing technology). One way of assessing phage diversity and indirectly abundance is using viral metagenomics. Metagenomics is where the total viral component from a particular environment is collected and sequenced (e.g., gut metagenomes; Camarillo-Guerrero et al., Cell, 2021, 184:1098-1109). Metagenomics can be used to identify phages or phage genes of environmental significance, such as genome modifying enzymes that that specifically modify the genome of host cells. Together with sequence characterization, gene fragments that have predicted enzyme domains or motifs can identified, providing a great starting point for identifying an active genome modifying enzyme for research and clinical applications (e.g., gene therapy). The present one vector system then provides a high-throughput process to identify a useful enzyme,

In addition to bacteriophages, other sources of enzyme polypeptides may include cyanophage, archaeal viruses and animal associated phages.

In some embodiments, a genome modifying enzyme or enzyme polypeptide may be isolated and/or derived from a fungi such as phages that infect fungi, i.e., mycophages. Mycophages are viruses that infect fungi (e.g., Chytridiomycota, Zygomycota, Ascomycota and Basidiomycota). Representative examples of fungi include, but not limited to yeast, Candida (e.g., C. albicans), S. homoeocarpa, Ophiostoma novo-ulmi, C. parasitica, Aspergillus Endothia and Valsa.

In some embodiments, a genome modifying enzyme or enzyme polypeptide may be isolated and/or derived from a eukaryote. The eukaryotes may include non-mammals and mammals. The higher eukaryotes are preferably mammals including humans.

With the accumulation of more and more postgenomic data, computational methods have been used to study the function of proteins including enzymes within genomes. In particular, with the advent of high-throughput metagenomics, it became possible to uncover an unparalleled amount of polypeptides and fragments, including prediction of large datasets of novel polypeptides that may have enzymatic features. A number of databases of bacteriophages comprising large numbers of polypeptides that may have enzymatic function are publicly available.

### Recombinases

In some embodiments, the genome modifying enzymes are genome recombinases. DNA recombinases are widely used in multicellular organisms to manipulate the structure of genomes and to control gene expression. Recombinases may be isolated from archaea, bacteria, eukaryotes and viruses. These enzymes, e.g., derived from bacteria (e.g., bacteriophages) and fungi, catalyze directionally sensitive DNA exchange reactions between short (30-40 nucleotides) target site sequences that are specific to each recombinase. These reactions enable four basic genome modifications: excision/insertion, inversion, translocation and cassette exchange, which have been used individually or combined in a wide range of configurations to control gene expression. In some aspects, recombinases are site specific recombinases including serine recombinases, and tyrosine recombinases.

In some embodiments, recombinases are serine recombinases. Serine recombinases catalyze precise rearrangement of DNA sequence through site specific recombination at attachment sites (e.g., attP and attB sites). Members of serine recombinases or resolvase/invertase superfamily include small serine recombinase, large serine recombinases and serine integrases. Most of recombinases have a catalytic domain at the N-terminus, followed by a small, helix-turn-helix (HTH) DNA-binding domain.

Exemplary serine recombinases include SsolSc 1904b, Sscol1904a, Aarn, MjaMJ1004, Pab, SsolC1913, HpyIS607, MceRv0921, MtuRv0921, MtuRv2979c, MtuRv2792c, MtuISY349, MtuRv3828c, SauSK1, spy, EcoTn21, Mlo92, EcoTn3, lla, Cpe, Sau, SK41, Bme, Ran, Y4CG, Rhiz, Sar, pNL1, pje, Xan, Pae, Xca Req, ISXc5, Tn917, Bme53, resolvase, γδ resolvase, Gin and Hin invertases, ISXc5, Tn5044 resolvases, ΦC31 integrase, Cgl, ppsTn55501, Aac, Rrh, Xfa911, Xfa910, pMER05, MuGIn, Bja, Y4bA, ΦRv1, R4, SCD78.04c, SC2E137, SC3C8.24, TndX, SPBe2, Spn, TnpX, Sau CczA, ΦPC1, A118, Cac1956, Cac1951, SauCerB, Φ105, SCH10.38c, EcoYBCK SCC88.14, etc.

The large serine recombinases (LSRs) are encoded in temperate phage genomes or on mobile elements that precisely cut and recombine DNA in a highly controllable and predictable way. The LSRs contain amino-terminal catalytic domain and larger carboxyl-terminal regions that range in size from ˜300 residues to ˜550 residues. The C-terminal region of the LSRs is comprised of multiple structural domains and is responsible for coordination of unique LSRs activities. The LSR C-terminal region is composed of two structural domains: a mixed alpha/beta DNA-binding “recombinase domain” linked to an unusual DNA-binding zinc ribbon domain. In phage integration, the LSRs act at specific sites, the attP site in the phage and the attB site in the host chromosome, where cleavage and strand exchange lead to the integrated prophage flanked by the recombinant sites attL and attR.

Systematic discovery and classification of large serine recombinases have been performed be several research groups, which characterize and predict a large number of enzyme polypeptides having catalytic domains and other structural features of LSRs, e.g., Camarillo-Guerrero et al., Cell, 2021, 184:1098-1109; and Durrant et al, 2021, doi: https://doi.org/10.1101/2021.11.05.467528; the contents of each of which are incorporated by reference herein in their entireties. The one vector system of the present invention can be used to further identify, from those enzyme polypeptides, active LSRs with DNA modification activities.

Recombinases also include tyrosine recombinases (TRs), a large group of enzymes that perform site-specific recombination in a manner that involves a tyrosine residue in the recombinase forming a covalent protein-DNA linkage in the reaction intermediate. Tyrosine recombinases break and rejoin single strands in pairs and form a Holliday junction intermediate. Phages and transposons encode specific TRs to promote their own integration and transfer in bacterial genomes. The subgroups of TRs include but are not limited to, P2, Xer, XerCD, IntKX, BPP-1, SXT, Cand, TnpA, Arch1, Myc, Cyan, TnpR, Arch2, RitA, RitC, integrin, Brujita, RitB, CTnDOT, Des, Lambda, Clost, Burk and Tn916.

Examples of tyrosine recombinases include the Flp recombinase from the 2u plasmid of Saccharomyces cerevisiae (as well as the thermostable variant of Flp (e.g., Flpe) the Cre recombinase of bacteriophage, the B2 recombinase from the pSB2 plasmid of Zygosaccharomyces bailii, the B3 recombinase from the pSB3 plasmid of Zygosaccharomyces rouxii, the KD recombinase from the pKD1 plasmid of Kluyveromyces drosophdarum, the KW recombinase from the pKWS1 plasmid of Kluyveromyces wallii, the R recombinase from the pSR1 plasmid of Zygosaccharomyces rouxii, the SM recombinase from the pSM1 plasmid of Zygosaccharomyces fermentati, the TD recombinase from the pTD1 plasmid of yeast Torulaspora delbrueckii, λ Int, and others.

The present one vector system can be used to screen extended datasets of TR sequences from prokaryotic genomes. By sequence analysis and classification, numerous novel tyrosine recombinase polypeptides, based on TR specific characteristics, such as distinctive structural features (e.g., ore-binding (CB) domain and catalytic domain) are predicted in genome sequence data of many bacteria. An exemplary dataset comprising more than a thousand enzyme polypeptides having tyrosine recombinase structural feature is ACLAME database (Boyd et al., 2009, Trends MicroBiol., 17:47-53; the contents of which are incorporated herein by reference in their entireties).

In some embodiments, the present one vector system is used to screen chimeric recombinases which are engineered to include a recombinase catalytic domain module and an exogenous DNA binding domain module (e.g., chimeric serine recombinases and chimeric tyrosine recombinases). The activity and specificity of chimeric tyrosine/serine recombinase can be examined using the present one vector system.

### Transposases and Transposable Elements

DNA transposase is an enzyme that binds to the end of a transposon and catalyzes its movement to another part of the genome by a cut and paste mechanism or a replicative transposition mechanism. A DNA transposase is often encoded in a transposon sequence. As used herein, the term “transposon” refers to a transposable element (TE), i.e., a nucleic acid sequence, which can change its position within a genome, sometimes creating or reversing mutations and altering the cell's genetic identity and genome size. Transposable elements make up a large fraction of the genome and are responsible for much of the mass of DNA in a eukaryotic cell. Transposons are very useful to researchers as a means to alter DNA inside a living organism. Their importance in personalized medicine is becoming increasingly relevant as well. There are at least two classes of TEs: Class I TEs or retrotransposons generally function via reverse transcription, while Class II TEs or DNA transposons encode the protein transposase, which they require for insertion and excision, and some of these TEs also encode other proteins.

Exemplary transposases include Transposase (Tnp) Tn5 family, Tc1/mariner superfamily (e.g., sleeping beauty transposon), piggyback (PB) transposases, and Tol2 transposable elements and transposases, Tn3 family, Tn7 family and Tn402 family. TEs exit commonly in many species and several datasets have been developed to detect, collect and annotate TEs from genome wide sequence data. The present one vector system is useful to screen and identify a specific transposase and its genomic modification activities from numerous transposase polypeptides in the datasets. Some available transposable elements datasets include, but are not limited to, RepetDB (accessible at urgi.versailles.inra.fr/repetdb) and DFAM (https://dfam.org/browse).

Retrotransposons (also called Class I transposable elements or transposons via RNA intermediates) and other types of TEs can be identified using the present one-vector system. The three major retrotransposon orders are long terminal repeat (LTR) retrotransposons, long interspersed elements (LINEs), and short interspersed elements (SINEs). Retrotransposons propagate via a copy-and-paste amplification mechanism that has allowed them to accumulate in DNA, giving rise to the bulk of repeats in eukaryotic genomes). Exemplary retrotransposons include the Alu, L1, SVA, and mammalian-wide interspersed repeats (MIRs) families, Mobile LINEs are RNA polymerase II (Pol II)-transcribed autonomous retrotransposons of several thousand base pairs (bp). Mobile SINEs are RNA polymerase III (Pol III)-transcribed nonautonomous retrotransposons. The lengths of SINE family members generally range from 85 to 500 bp.

Examples of other types of TEs are the Foldback (FB) elements of Drosophila melanogaster, the TU elements of Strongylocentrotus purpuratus, and Miniature Inverted-repeat Transposable Elements.

### Engineered and Chimeric Genome Modifying Enzymes

As discussed herein, the one vector system can be used to identify an engineered genome modifying enzyme. Exemplary genome modifying enzymes include, CRISPR-Cas-derived genome editing agents-nucleases, base editors, transposases/recombinases and prime editors, chimeric recombinases by fusing the catalytic domain of a recombinase and a DNA binding domain. Any modular design approach, in which proteins with different functional properties are fused together, can be employed to develop engineered or chimeric enzymes. An engineered genome modifying enzyme may also be mutated, e.g., to increase its DNA modification efficacy and specificity.

### Applications and Methods of Use

Using the one-vector systems described herein, and using any methods and assays as described herein, the present invention provides applications for screening and identifying an active genome modifying enzyme.

In some embodiments, the present one vector system provides a simple system to identify an active enzyme from numerous predicted enzyme polypeptides from extended datasets created by genome sequences and computational sequence analysis and characterization. The one-vector system, in combination with the current sequencing technologies (e.g., NGS) enables screening hundreds to thousands of enzymes in one large experiment.

In some embodiments, the present one vector system can be used to identify an enzyme or polypeptide having genome modifying function from any metagenomes (e.g., human gut bacteriophage metagenome database), any publicly available databases. The computer predicted polypeptides having catalytic activities may be used to create a library of one vectors, each of which comprises a polynucleotide encoding an enzyme polypeptide and a predicted target site sequence and a unique identifier. The one-vector libraries are pooled together for screening and identifying an enzyme with genome modifying activity. For example, the system may use to screen databases of the proteins with known enzyme functions such as Protein Data Bank (PDB, www.rcsb.org; (Berman et al, 2000)), ICEberg (Dataset EV3; http://db-mml.sjtu.edu.cn/cgi-bin/ICEberg/; (Bi et al, 2012)), ACLAME (http://aclame.ulb.ac.be; (Leplae et al, 2010)), ISFinder (database on the insertion sequences of TRs, www-is.biotoul.fr; (Siguier et al, 2012)), INTEGRALL (integron database, http://integrall.bio.ua.pt, (Moura et al, 2009)), and NCBI CDD (Marchler-Bauer et al, 2017) and other metagenomics discussed in this disclosure.

In accordance, the invention provides a method for determining which of a plurality of genome modifying enzymes is the most specific, said method comprising the steps of any one of the methods described herein.

In a related aspect, the invention provides a method for characterizing a genome-wide activity of a genome modifying enzyme, the method comprising the steps of any one of the above methods. In one specific embodiment, the genome wide activity includes intended on-target activity and/or unintended off-target activity.

In some aspects, the present one-vector system can be used to identify a target site sequence (e.g., a recognition sequence) that is recognizable by an enzyme that can modify a nucleic acid sequence (e.g., DNA) in a genome (e.g., in a cell or an organism). In accordance, the single integrated vector of the present one-vector system comprises a polynucleotide encoding the enzyme and a unique target site sequence and a unique identifier that correlates to the unique target site sequence in the vector. The target site may be a naturally occurring target site sequence, a predicted target site sequence, or an engineered target site for said genome modifying enzyme. Engineering methods include, but are not limited to, rational design and various types of selection. Rational design includes, for example, using databases comprising individual target site sequences of the same enzyme family.

As non-limiting examples, the one-vector system is used to screen and identify the most efficient attP/attB recognition sequence for a specific LSR.

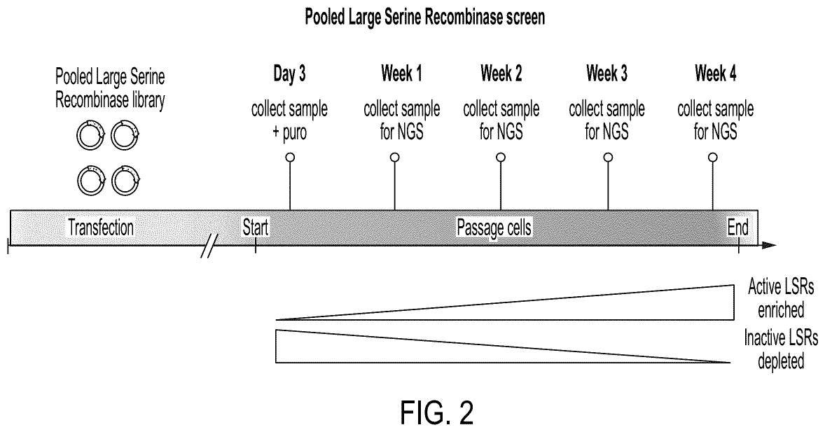

In one exemplary embodiment, the present method of identifying a genome modifying enzyme comprises: i) transfecting into cells a plurality of vectors, each of which comprises a nucleotide sequence encoding a unique genome modifying enzyme, a target site sequence that is recognizable by the unique genome modifying enzyme in the vector and a unique identifier that correlates to the genome modifying enzyme in the vector; ii) detecting genome modification activities in the transfected cells; iii) identifying the genome modifying enzyme by identifying the unique identifier in the transfected cells in which the genome modification activities are detected. FIG. 2 illustrates a representative process to identify a LSR using the one-vector system. In combination with the sequencing technology (e.g., NGS), the system can effectively identify an active enzyme.

### Compositions and Kits

In another aspect the disclosure provides, among other things, compositions and kits comprising the one vector system as described herein.

In some embodiments, the present invention provides compositions comprising one or more one vectors as described herein. The composition may further comprise one or more cell lines for transfection with the vectors.

In some embodiments, the present invention provides kits for use in the methods of the invention. The kits can include, for example, one or more of the following components: one or more one vectors, one or more cell lines, and reagents for cell transfections. Optionally, the kits may comprise a sequencing spacer, primers that bind to the sequencing spacer, and/or a container, and/or instructions for use in any one or more methods described herein.

These and other aspects of the present invention will be apparent to those of ordinary skill in the art in the following description, claims and drawings.

### EXAMPLES

### Example 1: Preparing an Integrated Vector

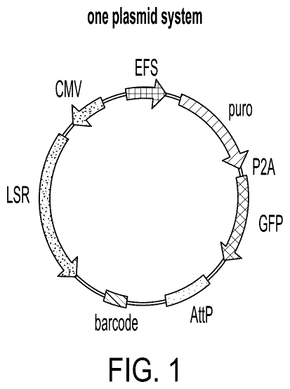

As a representative example, a vector for identifying a large serine recombinase (LSR) was designed and generated. As shown in FIG. 1, the integrated vector includes a nucleic acid sequence that encodes a LSR and a sequence including about 300 bp upstream of the LSR coding sequence from the phage genome and about 300 bp downstream of the LSR coding sequence from the phage genome. A DNA barcode having 20 nucleotides is added to the 3′ end of the recognition site (i.e., an attP site).

### Example 2: Processes for Identifying a Serine Recombinase Using one-Vector System

As illustrated in FIG. 2, the identification process, in a typical assay includes: transfecting cells with a plurality of one-vectors as described herein in Example 1. The transfected cells are cultured using standard procedures. After one week of the transfection, cells are harvested and tested for genomic modifications using, for example, next-generation sequencing (NGS). Optionally, the process is repeated one or more times to enrich the pooled enzymes that have DNA modification function.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A method for identifying a genome modifying enzyme using a one-vector system, wherein the one vector system comprises a vector comprising a nucleotide sequence encoding a genome modifying enzyme, a target site sequence that is recognizable by the genome modifying enzyme, and a unique identifier that correlates to the genome modifying enzyme.

2. The method of claim 1, wherein the genome modifying enzyme includes serine recombinase (e.g., large and small serine recombinases), tyrosine recombinase (e.g., Flp, λ-integrase, and Dre), retrotransposon (e.g., LTRs, LINEs and SINEs), DNA transposase, nuclease, chimeric genome modifying enzyme, and the like.

3. The method of claim 1 or 2, wherein the target site sequence is a native target site sequence, or an engineered target site sequence that is recognizable by the genome modifying enzyme in the same vector.

4. The method of any one of the preceding claims, wherein the unique identifier comprises a nucleotide sequence of about 5-30, 5-20, 8-30, 8-20, 10-30, or 10-20 nucleotides in length.

5. The method of any one of the preceding claims, wherein the unique identifier comprises a nucleotide sequence of about 20 nucleotides in length.

6. The method of any one of the preceding claims, wherein the vector further comprises a reporter.

7. The method of claim 6, wherein the reporter is a fluorescent protein or a variant thereof.

8. The method of claim 7, wherein the reporter is a green fluorescent protein (GFP) or a variant thereof.

9. The method of any one of the preceding claims, wherein the vector comprises a promoter sequence.

10. The method of claim 9, wherein the promoter is a constitutive or inducible.

11. The method of any one of the preceding claims, wherein the vector further comprises a selective marker.

12. The method of claim 11, wherein the selective biomarker is an antibiotic resistance gene (e.g., Puromycin).

13. The method of any one of the preceding claims, wherein the vector is a non-viral vector.

14. The method of claim 13, wherein the non-viral vector is a plasmid, a cosmid, an artificial chromosome vector, or the like.

15. The method of any one of claims 1-12, wherein the vector is a viral vector.

16. The method of claim 15, wherein the viral vector is an adeno-associated virus (AAV) based vector, an adenovirus based vector, a retrovirus based vector, a lentivirus based vector, a herpesviral vector, a rabies viral vector, or the like.

17. The method of any one of the preceding claims, wherein the genome modifying enzyme is a serine recombinase.

18. The method of claim 17, wherein the serine recombinase is a large serine recombinase (LSR).

19. The method of claim 18, wherein the LSR is from a bacteriophage.

20. The method of claim 18 or 19, wherein the target site sequence comprises an attP recognition site sequence that is derived from a sequence with about 200-300 bps downstream of the coding sequence of the LSR in the phage genome.

21. The method of claim 18 or 19, wherein the target site sequence comprises an attP recognition site sequence that is derived from a sequence with about 200-300 bps upstream of the coding sequence of the LSR in the phage genome.

22. The method of claim 18 or 19, wherein the target site sequence comprises a pseudo attP recognition site sequence that is recognizable by the LSR.

23. The method of claim 18 or 19, wherein the target site sequence comprises a corresponding attB recognition site sequence or a pseudo attB recognition site sequence that is recognizable by the LSR in the vector.

24. The method of any one of claims 19-23, wherein the recognition site sequence comprises about 30-75 nucleotides.

25. A method for identify a genome modifying enzyme for genome modification comprising:
i) transfecting into cells a plurality of vectors, each of which comprises a nucleotide sequence encoding a unique genome modifying enzyme, a target site sequence that is recognizable by the unique genome modifying enzyme in the vector and a unique identifier that correlates to the genome modifying enzyme in the vector;
ii) detecting genome modification activities in the transfected cells; and
iii) identifying the genome modifying enzyme by identifying the unique identifier in the transfected cells in which the genome modification activities are detected.

26. The method of claim 25, wherein the genome modification activities are detected about 2 days, 3 days, 5 days, 1 week, or 2 weeks after transfection.

27. The method of claim 25 or 26, wherein the genome modification is detected by next-generation sequencing (NGS).

28. The method of any one of claims 25-27, wherein the cells are mammalian cells.

29. The method of claim 28, wherein the cells are human cells.

30. The method of any one of claims 25-29, wherein the unique identifier in each vector comprises a nucleotide sequence of about 5-30, 5-20, 8-30, 8-20, 10-30, or 10-20 nucleotides in length.

31. The method of claim 30, wherein the unique identifier comprises a nucleotide sequence of about 20 nucleotides in length.

32. The method of any one of claims 25-31, wherein the target site sequence is a native target site sequence, or an engineered target site sequence that is recognizable by the genome modifying enzyme in the same vector.

33. The method of any one of claims 25-32, wherein the vector further comprises a reporter.

34. The method of claim 33, wherein the reporter is a fluorescent protein, or a variant thereof.

35. The method of any one of claims 25-34, wherein the vector comprises a promoter sequence.

36. The method of claim 35, wherein the promoter is a constitutive or inducible.

37. The method of any one of claims 25-36, wherein the vector further comprises a selective marker.

38. The method of claim 37, wherein the selective marker is an antibiotic resistance gene (e.g., Puromycin).

39. The method of any one of claims 25-38, wherein the vector is a non-viral vector, or a viral vector.

40. The method of claim 39, wherein the vector is a non-viral vector selected from plasmid, cosmid, artificial chromosome vector and the like.

41. The method of claim 39, wherein the vector is a viral vector selected from the group consisting of adeno-associated viral vector, adenoviral vector, retroviral vector, lentiviral vector, herpesviral vector, rabies viral vector and the like.

42. The method of any one of claims 25-41, wherein the genome modifying enzyme includes serine recombinase (e.g., large and small serine recombinases), tyrosine recombinase (e.g., Flp, λ-integrase, and Dre), retrotransposon, DNA transposase, and the like.

43. The method of claim 42, wherein the genome modifying enzyme is a serine recombinase.

44. The method of claim 43, wherein the serine recombinase is a large serine recombinase (LSR)

45. The method of claim 44, wherein the LSR is from a bacteriophage.

46. The method of claim 44 or 45, wherein the target site sequence comprises an attP recognition site sequence that is derived from a sequence with about 200-300 bps downstream of the coding sequence of the LSR in the phage genome.

47. The method of claim 44 or 45, wherein the target site sequence comprises an attP recognition site sequence that is derived from a sequence with about 200-300 bps upstream of the coding sequence of the LSR in the phage genome.

48. The method of claim 44 or 45, wherein the target site sequence comprises a pseudo attP recognition site sequence that is recognizable by the LSR.

49. The method of any one of claims 46-48, wherein the cells comprise a corresponding attB recognition site sequence, or a pseudo-attB recognition sequence that is recognizable by the LSR.

50. The method of claim 44 or 45, wherein the target site sequence in the vector comprises a corresponding attB recognition site sequence that is recognizable by the LSR.

51. The method of claim 44 or 45, wherein the target site sequence in the vector comprises a pseudo-attB recognition sequence that is recognizable by the LSR.

52. The method of claim 50 or 51, wherein the cells comprise a corresponding attP recognition site sequence, or a pseudo-attP recognition sequence that is recognizable by the LSR

53. A one vector system for identifying a genome modifying enzyme, wherein the vector comprises a nucleotide sequence encoding a genome modifying enzyme, a target site sequence that is recognizable by the genome modifying enzyme in the vector and a unique identifier that correlates to the genome modifying enzyme in the vector.

54. The one vector system of claim 53, wherein the vector further comprises a reporter gene.

55. The one vector system of claim 53 or 54, wherein the vector comprises a promoter sequence.

56. The one vector system of any one of claims 53-55, wherein the unique identifier comprises a nucleotide sequence of about 5-30, 5-20, 8-30, 8-20, 10-30, or 10-20 nucleotides in length.

57. The one vector system of any one of claims 53-56, wherein the vector comprises a selection marker.

58. The one vector system of any one of claims 53-57, wherein the system comprises a plurality of vectors, each of which comprises a nucleotide sequence encoding a unique genome modifying enzyme, a target site sequence that is recognizable by the unique genome modifying enzyme in the same vector and a unique identifier that correlates to the genome modifying enzyme in the same vector.

59. A method for identifying a recognition site sequence for a recombinase using a one vector system wherein the method comprising,
i) transfecting into cells a plurality of vectors, each of which comprises a nucleotide sequence encoding the recombinase and a unique recognition site sequence and a unique identifier that correlates to the unique recognition site sequence;
ii) detecting recombination activity in the transfected cells; and
iii) identifying the recognition site sequence by identifying the unique identifier in the transfected cells in which the recombination activity is detected.

60. The method of claim 59, wherein the recombinase is a large serine recombinase (LSR).

61. The method of claim 60, wherein the recognition site is an attP site sequence, or an attB site sequence.

62. The method of claim 61, wherein the recognition site sequence is a mutated attP site sequence or a mutated attB site sequence.
